(19) United States
(12) Patent Application Publication
(10) Pub. No.: US 2022/0001109 A1
(43) Pub. Date: Jan. 6, 2022

(54) SYRINGE ACCESSORY DEVICE AND METHODS OF USE

(71) Applicant: ERIC MICHAEL SIMON, SALT LAKE CITY, UT (US)

(72) Inventor: ERIC MICHAEL SIMON, SALT LAKE CITY, UT (US)

(21) Appl. No.: 17/289,439

(22) PCT Filed: Nov. 27, 2019
(86) PCT No.: PCT/US2019/063780

§ 371 Date: Apr. 28, 2021

Related U.S. Application Data
(60) Provisional application No. 62/772,974, filed on Nov. 29, 2018.

(51) Int. Cl. A61M 5/31; A61M 5/315

(52) U.S. Cl. CPC A61M 5/3137; A61M 2005/3139; A61M 5/31505

(57) ABSTRACT

A syringe accessory device which provides ergonomic advantage by allowing use of adductive thumb and finger force during aspiration, comprising a unitary structure, and configured for ease of attachment to a typical syringe. The accessory is comprised of three main sub-structures, contiguously formed, namely a plunger flange attachment, a syringe barrel attachment, and a linking element between the two attachments.

### BACKGROUND—FIELD OF INVENTION

This invention relates to syringe accessory devices, specifically to those devices which provide the user an ergonomic advantage during aspiration. This invention further relates to syringe accessories which may be mated to the syringe during production, or afterwards prior to or during use. This invention also relates to methods of use of the syringe accessory and the syringe to which it is attached.

### BACKGROUND—DESCRIPTION OF PRIOR ART

Many embodiments of syringes have been developed with built-in features to assist the user during aspiration (filling) of contents by providing a mechanical advantage, typically by allowing the users fingers and thumb to approximate or adduct (fingers and thumb “coming together”) in a motion similar to that used for ejection of contents. Examples of such syringes can be seen in U.S. Pat. No. 7,967,793 to Sibbitt, U.S. Pat. No. 463,924,893 to Schweblin, and U.S. Pat. No. 6,231,550 to Laughlin. At least one embodiment of syringe, exemplified in U.S. Pat. No. 3,325,061 to Ellsworth, has been comprised to include a slide component which allows thumb force to both aspirate and eject contents into or out of the syringe. Additionally, adaptive accessory devices have been developed to be secondarily fitted to existing “off-the-shelf” syringes to provide a similar action and benefit. It is the latter—accessories comprised to assist the user of a typical pre-existing syringe during aspiration—to which this invention relates.

As stated, Syringe Aspiration Accessories (SAAs) are known in the art. SAAs typically provide functionality to the user by increasing ergonomic advantage—applied force, hand position, grip security, single-handed use, etc.—when using the syringe. SAAs are known which increase the user's finger and/or thumb purchase on the syringe barrel and/or plunger; for example, ring-like structures comprised to fit on either the barrel or the plunger flange resulting in what is commonly known as a “control syringe” [Caillouette U.S. Pat. No. 5,246,011]. Other SAAs have been proposed which fit onto the syringe and allow the user to apply a pistol or trigger grip, and thereby achieve increased ejection pressure [De Santis U.S. Pat. No. 5,469,860].

A known style of SAA utilizes features which allow increased applied force with single-handed operation, and thus increase negative pressure within the syringe. Aspiration of the syringe (i.e., retracting the plunger outward from the barrel to achieve negative pressure within the barrel and thereby drawing external liquid or gaseous contents into the barrel) is commonly a two-handed procedure: The user holds the barrel in one hand and, using the fingers or fingers-and-thumb of the opposite hand on the plunger flange, pulls the plunger outward from the barrel. This procedure may be difficult to perform if one hand is required to perform an alternate function, such as holding a catheter or applying pressure at a needle insertion site or holding a fluid container such as an IV drip bag, and cannot assist in grasping the syringe. In such cases, the user is generally required to withdraw the plunger using only a single hand, typically by holding the syringe barrel with the fingers in a power grip and then applying force to the bottom of the plunger flange using the back of the thumb. This action is problematic for several reasons: First, abduction/extension of the thumb (moving the thumb away from the index and middle fingers, in a reverse action of that typically used to eject contents) may provide only 25-35% of the peak force generated by adduction/flexion [Inventor's observation, at a fingers-thumb separation distance of 50 mm: 1.8 max kgf vs. 6.2 max kgf]. Second, persons with small hands are challenged to achieve the necessary length of motion which may be required when single-handedly aspirating a larger syringe, e.g. 30 cc, 60 cc, and greater, with the back or tip of thumb. Third, common syringes do not generally provide grip or registration features on the underside of the plunger flange resulting in what may be an insecure contact of the thumb on the flange. Other single-handed aspiration grip-types exist. For example, if the syringe is held vertically (needle up), as is the case when drawing fluid into it from a dosing vial, the barrel is typically held with the index and middle fingers and the thumb, and the plunger is contacted by the ring and/or pinky fingers and retracted downward to draw in fluid. This action however provides no better ergonomic or mechanical advantage to the user than aspiration using the back of the thumb.

Various embodiments of SAAs have been proposed. These devices may be incorporated into the syringe itself (thus requiring use of various features or modifications of existing features of the syringe during manufacture to accommodate the adaptive components) [Taylor U.S. Pat. No. 3,990,446], or alternately may be separate devices designed to attach to the previously produced off-the-shelf syringe at some point after production [Bertocci U.S. Pat. No. 9,067,023]. As stated, control syringes having loops, parallel flanges, or other finger and thumb grasping means are well known. However, a control syringe still relies on abduction (extension) of the thumb which is a low-force means of withdrawing the plunger from the barrel. To overcome the mechanical disadvantage of abducting the fingers and thumb, spring components have been added to syringes, typically between the plunger flange and the open end of the barrel, to provide spring-powered plunger retraction. This assemblage has the disadvantage of requiring an additional, usually metallic spring component as well as putting retraction force on the plunger in the resting position—two factors that are generally undesired.

Other embodiments of SAAs rely on providing the user with features or structures by which the user may apply flexural (i.e., adducting) forces with the fingers and thumb to achieve aspiration by forcing the retraction of the plunger from the barrel. These embodiments generally provide a mechanical attachment between the plunger flange and a mating structure on the accessory such that adductive motion of the thumb (moving the thumb toward the palm of the hand) may be used for both ejection and aspiration of contents [Gammon U.S. Pat. No. 6,719,735]. Alternately, embodiments exist that allow an alternative motion—e.g., the “sliding” action of the thumb while the barrel is cradled by the fingers—to provide fine control to both aspiration and ejection of the syringe contents, such as may be useful during low-angle-of-entry dermatological procedures [Nerney U.S. Pat. No. 7,118,556].

The shortcomings of these “compressive-style” accessory devices are known. They are usually comprised of two or more components, which increases cost and complexity, and may increase the risk of failure or poor performance [Fulk U.S. Pat. No. 58,140,231]. Some of these accessories add a component which acts as a gantry or tower over the plunger which increases the length and unwieldiness of the syringe+accessory assemblage [Gammon ibid, Houghton U.S. Pat. No. 5,135,511]. Some require a structural element of the accessory to be looped over the ported end of the syringe prior to attachment—a problematic action if the syringe orifice is already connected to a needle or tubing [Tartaglia U.S. Pat. No. 4,484,915]. One prior art SAA, though technically molded as a single piece, requires the use of living hinges and precision snap fits, necessitating a complex action to affix the accessory to the syringe [Bertocci ibid]. Many are not true accessories but are intended to be incorporated onto or within a modified syringe during manufacture thus making them unusable as a retro-fittable device for use on existing typical syringes [Ellsworth U.S. Pat. No. 3,325,061, Gandarias US20040073172, Haber U.S. Pat. No. 5,582,595]. Most SAAs lack broad applicability; i.e. they are configured to attach to a specific syringe with a particular barrel diameter and plunger flange diameter.

### SUMMARY—OBJECTS AND ADVANTAGES

The proposed syringe aspiration accessory is comprised to address the various problems with the existing range of such devices. Accordingly, several objects and advantages of the present invention are as follows:

(1) Legacy/Modularity. The accessory is designed to readily fit onto existing (aka prior-produced, stock, off-the-shelf) syringes. Syringes are highly evolved devices and are now generally produced in completely automated fashion, particularly those intended to be disposable (which is now the major fraction, by number of units made and sold worldwide). Thus to modify features on the syringe, and associated changes to tooling, molds, and assembly machinery/procedures in order to produce the syringe+accessory as an integrated device can be problematic from a manufacturing cost and inventory standpoint. It is a significantly more practical approach to either mate the accessory to a prior-produced syringe at the site of production/assembly and market it as an integrated device, or perhaps more commonly to provide the accessory as a separate product which can readily be mated to the syringe by the user at the time of use. Several prior art accessory devices meet this criterion but are problematic in various ways—too much complexity, high part count, poor ergonomics, etc.

(2) Attachment/Mating. In both cases—mating during manufacture or mating at time of use—the design of the accessory device should be implemented so as to afford ease of attachment of the accessory to the syringe; ideally, the accessory can be mated (and if required unmated) to the syringe quickly and easily. In the case of attachment during manufacture, especially for disposable syringes, the accessory preferably is embodied so as to securely engage the required features of the syringe barrel and plunger in an action amenable to the use of automated equipment for this procedure. This generally requires pick-and-place actions and motions of the assembly machine—simple linear and rotational motions, ease of grasping, features facilitating good machine vision (for grasping and orientation), minimal outwardly projecting features to facilitate loose packing in the supply bin (and thus ease of grasping one unit at a time), and minimizing the overall number of motions the machinery is required to perform to achieve precise and reliable mating. Ideally, a simple linear lateral-to-medial motion of the accessory onto the syringe is preferred, especially if the syringe can be positioned on its side (i.e., horizontally) in an assembly fixture. In the case of the accessory being applied at the time of use, it is preferable again to minimize motions and thereby increase ease of mating and while facilitating the possibility of one-handed mating between the accessory and the syringe.

Simple linear or rotation motions are again desired and ideally the accessory can be readily affixed to the syringe using one hand.

(3) Simplicity. More complex mechanical means of affixing the accessory to the syringe are to be avoided: Screw-, belt/band-, or hinge-type fitments (living or mechanical) are not desired. Such mechanical fixation means increase cost and complexity, induce problematic ergonomics, and reduce reliability. Additionally, structures such as living hinges often require the use of a limited set of materials having specific characteristics to perform properly, thus restricting the scope of applicable materials. For these reasons, simple snap-fits are generally desired.

(4) Part Count. The prior art predominantly shows accessory devices comprised of two, three, or more components, these components typically being assembled during manufacture. The use of multiple components and the requirement for one or more assembly steps again raises cost and complexity and often reduces reliability during use. In particular, increased cost works against the commonly desired goal of disposability. Additionally, a multi-component assembly may utilize one or more components that might preclude certain sterilization methods due to material or shadowing considerations. A unitary or single-piece construction of the syringe accessory is desired.

(5) Materials. The accessory should be preferably molded from common thermoplastic or thermoset polymers, or powdered metals, or 3-D printed in either plastic or metal to allow high production speeds with a high degree of feature detail. The production of molded or 3-D printed metal or glass-filled plastic accessories may facilitate durable-use which allows the accessory to be used multiple times, mating to and removing from disposable or reusable syringes as needed. Such durable-use accessories may be preferred for applications involving nonsterile dispensing syringes, like those used during adhesive or lubricant application as are commonly employed in the manufacturing and servicing of various products.

(6) Geometry. The physical features of the accessory are designed to simplify molding as much as possible. Undercut and hollow features in molded parts usually require side-action components of a mold to slide or rotate out of the way before the part can be ejected from the mold, increasing cycle-time and part cost and reducing mold life and reliability. SAAs designed to be molded from A-B straight pull molds (with no side actions) are desired.

(7) Ergonomics. Syringes are commonly held in various grip-styles and at various orientations depending upon the nature of the procedure in which the syringe is being employed. It is therefore beneficial to be able to dexterously interact with the accessory in various ways and in various orientations during use. An ergonomically multi-functional/interactional accessory is desired.

(8) Visualization. Visualization is important during syringe procedures since a specific volume of gas or liquid is often required to be ejected or aspirated (and thus syringes, especially those used in medical procedures, are commonly provided with volumetric gradations printed or embossed onto the outer surface of the barrel). SAAs which do not interfere with visualizing the fluid line and/or leading edge of the piston seal are thus desired.

(9) Mating. If a syringe is in use prior to affixing the accessory, it may be necessary to avoid having to pass the accessory over the end of the syringe port (the I/O port of the syringe—luer, nozzle, etc) since it may already be attached to a needle, tubing, catheter, or other elongated component. Additionally, the sterility of the port or what is attached to it, e.g. needle, might be compromised should it be contacted. If a needle is affixed to the port, affixing an accessory over it may present an additional sharps/puncture risk. Thus, if fitment to the outer surface of the syringe barrel is a requirement of the accessory, such fitment should not require removal of a needle or length of tubing but instead should provide for them to be left unperturbed. Mating the SAA to the syringe barrel using structure(s) which do not pass over the syringe I/O port during attachment or removal is desired.

(10) Action. The interface between the SAA and the syringe should minimize friction and off-axis forces to increase efficiency and ease-of-use. Well designed elements and sub-features within the SAA to promote smooth and reliable operation are desired.

(11) Size. The overall size of the SAA is important in terms of production cost, required inventory/storage space, and bulkiness/unwieldiness during use and disposal. Similarly, a SAA which fits onto the syringe barrel in a manner which effectively lengthens the syringe assembly, e.g. Gammon U.S. Pat. No. 6,719,735, also may be problematic. A minimally-sized accessory is desired.

(12) Scalability. The form of the SAA should be such that it can be produced in different sizes to accommodate various syringe sizes and geometries. Likewise, the structures of the accessory should be easily modified so as to allow production of a range of accessories designed for use on different syringe geometries, such as is commonly seen between different manufacturers and syringes intended for specific tasks (e.g., drug injection, fluid aspiration, surgical procedures, vascular procedures, adhesive dispensing, etc.). SAAs capable of being produced in a range of sizes and shape variations are desired.

(13) Commonality. The accessory should be formed in a manner that allows the use of typical or commonly-used syringes. For example, a Becton-Dickinson 30 cc (1 oz) syringe with a luer-lock tip is a common type of disposable syringe and is used in many different types of medical and laboratory procedures. An accessory for this specific syringe (and syringes of similar geometry) should be able to be affixed without any modification of the syringe. If any adjustment is required, it should be minimal—e.g., very slight rotation or repositioning of the plunger. Conversely, if the plunger has previously been withdrawn to any position, the accessory should be able to be readily mated or unmated to the syringe in that position. An SAA capable of being affixed to a readily available syringe is desired.

(14) Universality. The geometries of the various syringes within a volumetric class, e.g. 30 cc/35 cc syringes, as produced by different syringe manufacturers can vary to some degree. For example, the outside diameter (OD) of the barrel of a 30 cc syringe produced by Covidien Monoject may be as large as 26.1 mm whereas that of a BD 30 cc syringe may be 24 mm. Similarly, the plunger flange diameter of the Covidien-Monoject is approximately 29.3 mm while that of the BD is approximately 25.9 mm. An accessory which can fit a range of syringe geometries within a volumetric class is desired.

(15) Anti-rotation/Registration. It may be preferable for the accessory to remain constrained to a plane of the syringe (front/back or side/side), simply moving linearly within that plane without rotation. An accessory comprised to constrain or limit twist (out of plane motion), particularly twist of the plunger in relation to the barrel, is desired.

(16) Removal. It may be useful or necessary to remove the accessory from the syringe. This would be the case when a durable-use accessory is being employed (e.g., removal after procedure prior to disposal of the syringe), or alternately if removal of the accessory facilitates some aspect of the procedure itself. Various geometric features within the accessory, such as ribs, flanges, lands, latches/catches, etc., might assist the user with removing the accessory from the syringe—i.e., disengaging one or more accessory-to-syringe fitments—by providing some sort of ergonomic advantage. Features and methods for assisting removal of the SAA from the syringe—in those situations where removal is useful or advised—are desired.

(17) Disposal. It would be preferred that any accessory designed for single-use be amenable to recycling. If incineration is required (as is common with medical disposables), the material(s) used in production of the accessory should be chosen to facilitate such means of disposal (e.g., little to no production of toxic waste gases). Additionally low or single part count is preferred to obviate disassembly and component separation prior to downstream processing. Embodiment and material composition of the accessory to facilitate appropriate disposal or recycling methods is desired.

(18) Sterilization. It may be necessary to sterilize the accessory during manufacture such that the accessory is received—either separately or pre-attached to the syringe—in a sterile condition (within sterile packaging). Conversely, the accessory may be sold in a nonsterilized condition and later packaged and sterilized prior to use. Different methods of sterilization may be applied by different packagers. Thus the ability of the accessory to withstand more than one method of sterilization and/or repeated sterilization cycles may be a desirable characteristic.

(19) Manufacturability. Material selection and use of an accessory geometry amenable to economical fabrication with that material via specific desired processes (e.g., injection molding, 3D printing, etc) is important. Simple, cost-effective manufacturing—for example, injection molding using A-B straight pull molds—is desirable.

Still further objects and advantages will become apparent from a consideration of the ensuing description and accompanying drawings.

### SUMMARY—EMBODIMENT OF THE INVENTION

The embodiment of the proposed syringe accessory device comprises three functional sub-structures connected in a unitary fashion: (1) A plunger flange attachment; (2) A sliding barrel attachment; and (3) A linking element between the two attachments.

This invention describes a syringe accessory device which provides ergonomic advantage by allowing use of adductive fingers-to-thumb force during aspiration, comprising a single unitary component, and designed for ease of attachment and detachment to an existing syringe.

### BRIEF DESCRIPTION OF THE DRAWINGS

FIGS. 1A-1C are front, side-sectional, and detail views of a typical 3-piece disposable syringe (30 cc luer-lock syringe, approximately 1:1 scale) [PRIOR ART].

FIGS. 1D and 1F are perspective and detail views of the same a typical 3-piece disposable syringe. FIG. 1E is a detail view of FIG. 1D showing a notched-collar alternative to the syringe [PRIOR ART].

FIGS. 2A, 2C-2E are front superior perspective views of a simplified one-piece syringe aspiration accessory in isolation and attached to a syringe (approximately 1:2 scale). FIG. 2B is a detail view of FIG. 2A of the plunger flange attachment.

FIG. 3A is a perspective view of the simplified syringe aspiration accessory mated to a syringe showing a typical grip (and an alternate thereof) used to aspirate contents into the syringe.

FIG. 3B is a perspective view of the simplified syringe aspiration accessory mated to a syringe showing a typical grip used to eject the syringe contents (normal syringe ejection).

FIG. 3C is a perspective view of the simplified syringe aspiration accessory mated to a syringe showing an alternate “cigar” grip used to aspirate contents into the syringe.

FIG. 3D is an alternate perspective view of the simplified syringe aspiration accessory mated to a syringe showing a “cradle” grip used to either aspirate contents into or eject contents out of the syringe, respectively using a thumb flexion or extension action.

FIGS. 4A, 4D-4E, and 4G-4H are perspective, front, side, and back views of a preferred one-piece syringe aspiration accessory in isolation. FIGS. 4B and 4C are detail views of FIG. 4A. FIG. 4F is a detail view of FIG. 4E.

FIG. 5A is a front view of the preferred syringe aspiration accessory of FIGS. 4A-4G affixed to a typical disposable syringe. FIG. 5B is a section view through line F-F of FIG. 5A. FIG. 5C is a section view through line G-G of FIG. 5A. FIG. 5D is an alternate section view through line G-G of

FIG. 5A wherein the notched-collar syringe of FIG. 1E is employed. FIG. 5E is a detail view of FIG. 5D. FIG. 5F is a section view through line H-H of FIG. 5A. FIG. 5G is a detail view of FIG. 5F.

FIG. 6A is a perspective view of an alternate preferred syringe aspiration syringe accessory comprised to affix to a range of plunger flange diameters. FIG. 6B is a detail view of FIG. 6A. FIGS. 6C-6E are rear detail views of variations of the alternate preferred syringe aspiration syringe accessory of FIG. 6A, showing various full reliefs. FIGS. 6F-6I are rear perspective detail views of other variations of the alternate preferred syringe aspiration accessory of FIG. 6A, showing various partial reliefs.

FIG. 7A is a perspective view of a different alternate preferred syringe aspiration accessory comprised to affix to a range of plunger flange diameters and plunger flange thicknesses. FIG. 7B is a detail view of FIG. 7A. FIG. 7C is a side view of FIG. 7B. FIG. 7D is a detail view of FIG. 7C. FIG. 7E is a front view of FIG. 7B. FIG. 7F is a detail perspective view of a variant embodiment of the preferred syringe aspiration adapter of FIGS. 7A-7E. FIGS. 7G and 7H are side and front views respectively of FIG. 7F.

FIG. 8A is a perspective front view of an alternate preferred syringe aspiration accessory comprised to provide improved viewing of the syringe's fill level when viewed from the rear of the PSAA. FIG. 8B is a perspective rear view of the alternate PSAA of FIG. 8A. FIG. 8C is a perspective rear view of the alternate PSAA of FIG. 8A attached to a syringe.

Reference Numerals

100 typical 3-piece syringe
110 syringe barrel
111 barrel bore (inside wall)
112 barrel outside wall
113 ported end of barrel
114 barrel I/O port
115 barrel open end
116 barrel top collar
1161 top collar finger flanges
1162 top collar annular flanges
1162B top collar detents
1163 top collar ribs
117 luer-lock fitting
118 plunger safety stop
119 volumetric gauge marks
120 syringe plunger
121 plunger shaft
122 plunger top flange
123 piston attachment fitting
124 plunger flange traction features
125 intermediate flange(s)
126 plunger ribs
130 piston
131 piston mating undercut
132 piston annular sealing lips
133 piston bottom face
200 simple syringe aspiration accessory (SSAA) embodiment
210 simple plunger flange attachment
211 top plate
212 side wall
213 bottom plate
220 simple syringe barrel attachment
221 simple finger tabs
230 simple linkage
231 simple thumb tabs
400 preferred syringe aspiration accessory (PSAA) embodiment
410 preferred plunger flange attachment (PPFA)
411 top plate
412 side walls
413 bottom plate
414 push tab
415 grip assist features
416 spacer ribs
417 PPFA mouth
4171 vertical edges
4172 lead-in taper
4173 retention taper
418 upper molding windows
420 preferred syringe barrel attachment (PSBA)
421 barrel stand-offs
422 PSBA mouth
423 force application features
423′ downward-offset force application features
423″ upward-offset force application features
424 thumb-assist flange
425 traction structures
426 PSBA reliefs
427 lower molding channels
430 preferred linkage
431 linkage stiffening ribs
432 linkage guide rib
433 linkage slots
434 traction grooves
435 starter wings
436 secondary thumb-assist flange
600 alternate-1 PPFA
601 side expansion reliefs
601′ top plate expansion reliefs A
601″ top plate expansion reliefs B
602 linkage expansion reliefs
603 partial top plate reliefs
604 contiguous linkage relief
700 alternate-2 PPFA
701 leaf spring
701′ flexed leaf spring
800 alternate-1 PSBA
801 expanded lower molding channel

### DETAILED DESCRIPTION—TYPICAL EMBODIMENTS

### PRIOR ART

### Typical 3-Piece Syringes

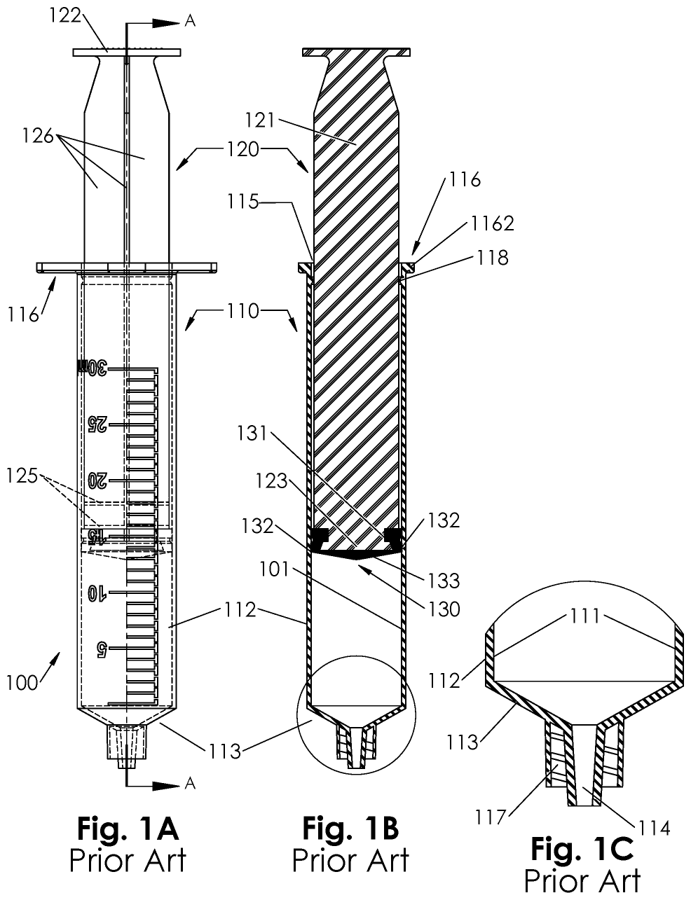

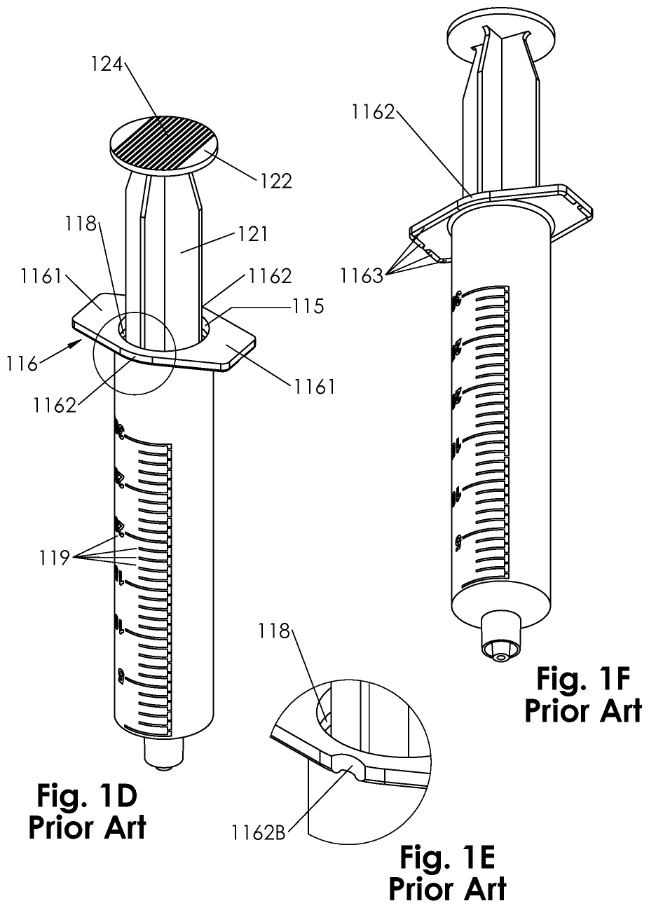

FIGS. 1A-1D are a typical 3-piece syringe 100 depicted in front, side-sectional, detail, and perspective views showing the essential structural elements of the device. Syringe 100 comprises three main parts: syringe barrel 110, syringe plunger 120, and piston (aka, piston or wiper) 130. [Note: 2-piece syringes are also common, especially for nonmedical dispensing applications, where the plunger 120 and piston 130 have been combined into a single unitary molded component. The piston of a 2-piece syringe is generally a circular flange-like element with a thin peripheral edge to provide a sliding seal on the inside surface (bore) of the barrel].

The barrel 110 has a cylindrical inner barrel bore 111 and a cylindrical barrel outside wall 112. The barrel has a ported end 113, comprising an input/output (I/O) port 114, and an open end 115 into which the plunger is inserted. The I/O port of the barrel may be configured in various ways to facilitate the attachment of needles or dispensing tips (e.g., luer-slip and luer-lock connections), tubing or catheters (e.g., catheter tip connections), conical (e.g., dispensing tip), or various other fluidic couplings. The syringe of FIG. 1 is depicted as having a luer-lock fitting 117, a common type of connector. The open end of the barrel 115 is typically a simple circular orifice having approximately the same or slightly larger inside diameter as the nominal ID of the barrel. Open end 115 typically has a top collar 116 which commonly comprises two barrel finger flanges 1161 for finger engagement during the ejection process, and short annular flanges 1162 contiguous with the finger flanges 1161 such that the top collar extends around the entire periphery of the barrel open end 115. The annular flanges 1162 may have small collar detents 1162B, as shown in FIG. 1E. Low profile collar ribs 1163 may be present on the periphery of the top collar to increase stiffness or provide finger purchase, such ribs being either continuous or segmented and generally located on the bottom surface of the top collar.

The bore of the barrel proximal to the open end 115 may comprise a safety stop 118 in the form of small ridges, rings, or bumps to help retain the plunger within the bore of the barrel (i.e., to prevent unwanted disengagement of the plunger from the barrel). It is common for the barrel outside surface 112 to comprise numbered volumetric gauge marks 119 for the user to determine the static volume of the syringe's contents, or the amount ejected or aspirated. Gauge marks 119 are typically printed on the barrel outside wall 112, but alternately may be embossed or molded upon the barrel outside wall.

The syringe plunger 120 is typically a 1-piece component comprising a shaft 121, a plunger top flange 122, and a piston attachment fitting 123. The plunger shaft 121 is commonly either circular, tubular, or cross-shaped in cross section (cross-shaped is common due to ease of molding, good stiffness, and reduced tendency to warp—see plunger ribs 126). The plunger top flange 122 is typically a disc of material (though other shapes are known) positioned on the upper end of the shaft to provide a contact surface for the thumb during ejection or a peripheral edge to grasp with the thumb and finger during two-handed aspiration. The diameter of the top flange 122 is generally larger than the nominal diameter of the shaft (or side-to-side extent of plunger ribs) to provide an undercut to facilitate grasping (as during two-handed aspiration). The upper surface of the top flange may be provided with traction features 124 such as low profile ribs, grooves, or bumps on the top surface to enhance grip and reduce the possibility of finger or thumb slippage. The piston attachment fitting 123 is commonly one or more circular flanges, smaller than the nominal OD of the plunger shaft 121, configured to engage an one or more undercuts in the piston 131 such that the piston 130 and the plunger 120 are securely engaged via annular snap-fit; other types of plunger-to-piston fitments are known, such as screw-fits. Other features such as intermediate flanges 125 may reside at various positions along the length of the plunger 120 to provide a stiffening or anti-warp function.

Piston 130 (shown in FIG. 1B as solid black) is generally comprised of a compliant material such as a thermoset or thermoplastic rubber, including but not exclusive to polyisoprene, neoprene, nitrile, thermoplastic elastomer, or silicone rubbers. The exterior surface of the seal 130 may have a slip coating or lubricant applied during manufacture to prevent binding or frictional dragging of the piston seal 130 on the barrel bore 111. Piston 130 is typically attached to the plunger 120 at the piston attachment fitting using at least one mating undercut 131 to engage the snap-fit. Additionally, the outer cylindrical surface of the piston is typically provided with one or more annular lips 132 to achieve a fluid-tight but smoothly sliding seal to the bore surface 111. The piston bottom face 133 is often conical or dome shaped to abut without gaps with the opposing internal surface of the ported end 113 of the barrel such that all fluid may be completely or near-completely ejected from the barrel at the end of the plunger stroke.

### Simplified Embodiment

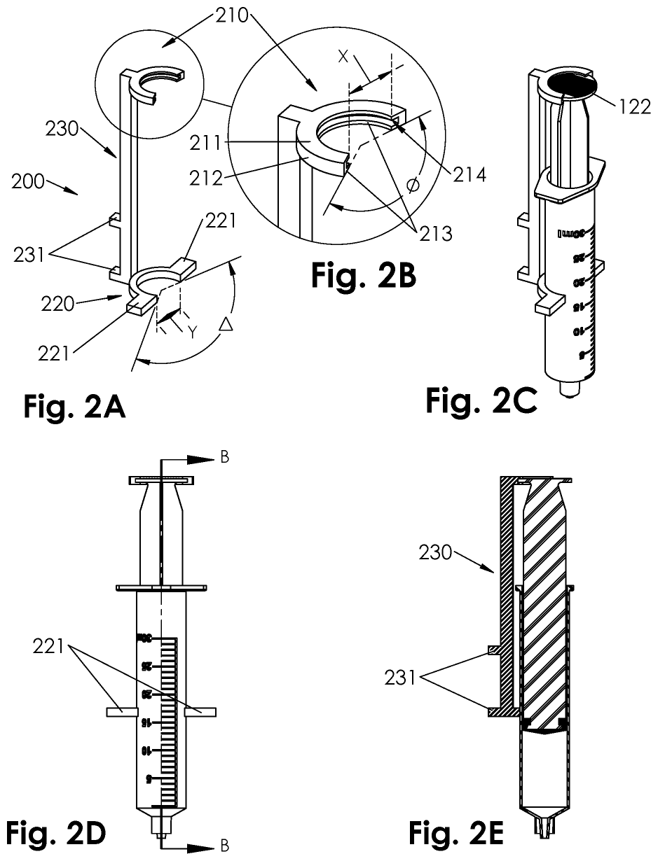

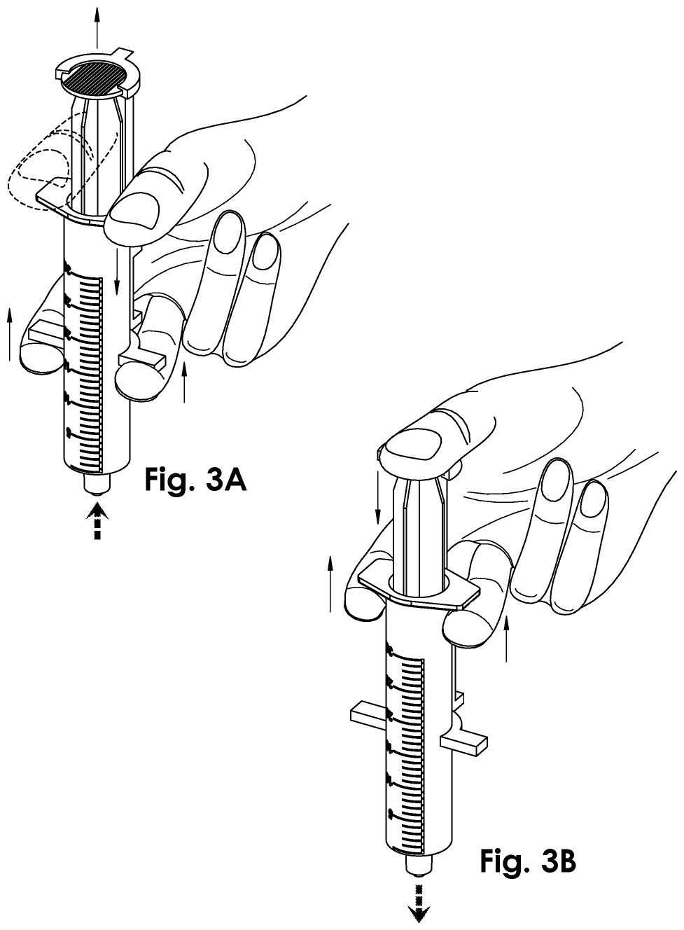

FIGS. 2A-2E show views of a of a Simple Syringe Aspiration Accessory (SSAA) 200. FIGS. 2C-2E show perspective, front, and sectional views of the SSAA 200 affixed to a typical syringe. Three main functional structures exist: A simple plunger flange attachment 210, a simple syringe barrel attachment 220, and a simple linkage 230 unitary with and spanning the two attachment structures.

The simple plunger flange attachment 210 (SPFA) is a fitment comprised of three main elements—a simple top plate 211, a simple side wall 212, and a simple bottom plate 213. In the simplified embodiment, the three elements are unitary and comprise a substantially C-shaped cross section with an internal mating groove 214 to securely affix to the plunger top flange 122, typically through a clamp type snap-fit (also known as a snap clamp). It is advantageous that this mating is easy to engage yet produces a secure fit, holding the plunger top flange and the flange attachment means 210 in a manner by which both upward or downward axial force may be transmitted. The fit may be irreversible (1-way) or for applications in which disengagement is desired, reversible. Ideally, the flange attachment means 210 can be affixed to the plunger flange 122 using a simple linear motion, e.g. the attachment means approximates and engages the plunger flange in a lateral-to-medial motion; conversely detachment can be achieved through the reverse motion. As is typical of sleeve-type snap-fits, the open side of the sleeve of the SPFA subtends an angle of less than 180 degrees (Ø) and thus the minimum jaw-to-jaw opening width (X) will be smaller than the OD of the cylindrical component (in this case the OD of the plunger flange) to which it is affixed. The dimensions of both Ø and X will be determined not only by the diameter of the plunger flange but also by the stiffness and lubricity of the materials used to form the SSAA 200, its cross-sectional geometry, and the the material used to form the syringe barrel 110, as well as the desired resistance to achieve snap engagement or disengagement of the SPFA 210 to the plunger top flange 122.

The simple syringe barrel attachment 220 (SSBA) is a cylindrical sleeve having an open side. The open side allows the the SSBA 220 to snap-fit onto the exterior wall of the barrel 112 using a lateral-to-medial motion (similar to the motion required for engaging the flange attachment means) in a C-clamp fashion. Connection of this sub-structure to the syringe barrel produces a secure yet axially sliding fit: The SSBA 220 fits onto the barrel without undesired diametral or off-axial looseness but can readily slide up and down the exterior wall of the barrel. Analogous to angle Ø and width X of the plunger flange attachment, angle Δ and width Y of the barrel attachment will be less than 180 degrees and smaller than the OD of the syringe barrel, respectively, to allow for a secure sliding fit onto the barrel. Likewise exact values of Δ and Y will be determined by the stiffness and lubricity of the materials employed, its form and cross-sectional geometry, and the desired force to engage and disengage the barrel attachment means onto or off of the barrel.

The barrel attachment means also may include one or more force application features for the user's fingers or thumb to engage—flanges, wings, tabs, loops, hooks, arches, ribs, ridges, and other finger engagement structures may be utilized depending upon the desired application and ergonomics of use. Force application features such as finger tabs 221, shown in FIG. 2A-C as simple rectangular posts, may be similar in size and shape to the finger flanges 1161 on the syringe barrel.

Simple linkage 230 is a substantially linear sub-structure spanning (or preformed and subsequently affixed to) peripheral locations of both the simple plunger flange attachment 210 and the simple barrel attachment 220. The cross-sectional shape of the simple linkage 203 is shown as being square or rectangular but other forms may be suitable, such as circular, ovoid, I-beam, H-beam, and others. The function of simple linkage 230 is to connect the two attachment means 210 and 220 such that when manual force is applied to one to induce movement, the other will move synchronously. Thus, for example, when upward force is applied by one or more fingers to the bottom surfaces of the finger tabs 221, linkage 230 will transmit the force to the simple plunger flange attachment 210 and to the plunger flange 122 to which it is attached, and thereby induce aspiration by retracting the plunger outward from the open end 115 of the barrel. Conversely, if force is applied to the top of the plunger flange attachment 210 by thumb or finger pressure, the plunger and piston will be forced into the barrel resulting in ejection of the syringe contents (e.g., as during injection of contents into a desired location), and the linkage 230 will force the motion of the barrel attachment 220 in a reverse fashion, down the syringe barrel 110 toward the ported end 113. Therefore, by applying force either to the barrel attachment 220 or the plunger flange attachment 210, fluids or gasses may be respectively aspirated into or ejected from the syringe using approximation (adduction, coming together) of the fingers and thumb for both procedures. This is advantageous since significantly higher net force can be generated when adducting (approximating or flexing) the thumb to the fingers as can be produced when abducting (deproximating or extending) the thumb from the fingers.

One or more other force application features may be positioned on the outer surface of the linkage 230 and/or barrel attachment 220. In FIGS. 2A and 2E, simple thumb tabs 231 are shown and are positioned to allow the syringe to be held in a cradle grip (also commonly known as a hook or bucket grip) with the fingers and the thumb used in a sliding fashion to either aspirate or eject contents.

Methods of Use—SSAA

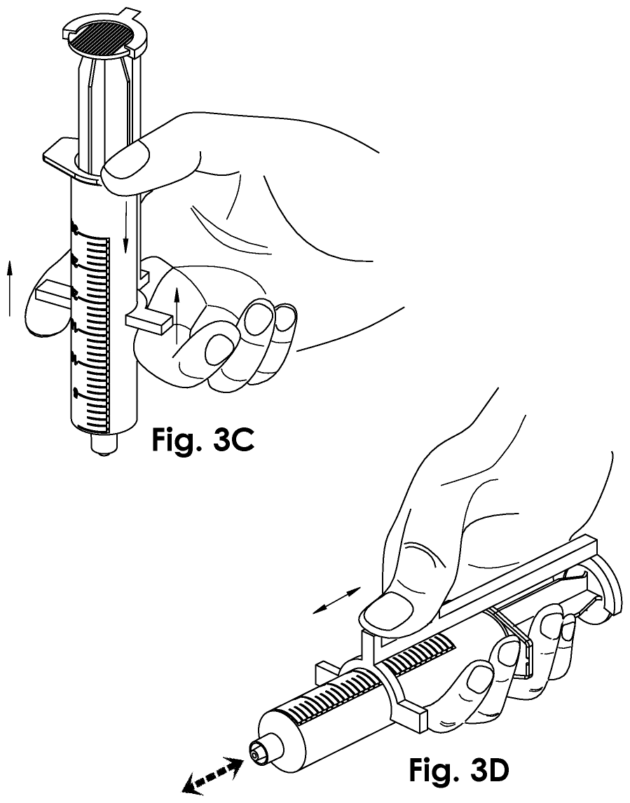

FIGS. 3A-3B show typical grip modes for aspiration and ejection, respectively, using the SSAA embodiment. To aspirate the syringe, the first and index fingers are placed on the bottom surfaces of the simple finger tabs of the SSAA and the thumb is placed on either of the syringe top collar finger flanges (alternative thumb position is shown in dashed line). As the fingers and thumb are drawn together (in a manner similar to the normal ejection action of the syringe), the plunger flange attachment will induce the plunger to withdraw and thereby aspirate contents into the syringe (FIG. 3A, dashed arrow). A variant of this grip (not shown) wherein the user applies the middle and ring fingers instead of the index and middle, may be useful for users with smaller hands to increase effective fingers-to-thumb reach.

If the fingers are placed in the normal ejection positions on the under surfaces of the syringe finger flanges and the thumb upon the top surface of the plunger flange attachment and/or plunger flange, contents will be ejected (dashed arrow) from the syringe as the fingers and thumb are adducted (FIG. 3B). Fingers-to-thumb adduction thus becomes applicable for both ejection and aspiration. It is seen that both aspiration and ejection utilize adduction of the fingers and thumb, thus generating roughly equal manual power for both functions while providing a secure, ergonomically favorable grip for the user. By regrasping the appropriate finger/thumb features, the action of the syringe can be readily reversed, which may for example be useful in procedures such as irrigations where aspiration and ejection of fluids can occur multiple times.

FIG. 3C shows use of an alternate grip (sometimes referred to as a “cigar” grip) to that shown in FIG. 3A wherein the index finger wrap around the barrel, the middle finger is curved beneath the finger tab of the SSAA, and the thumb is used to exert downward force on the barrel flange to induce aspiration. This grip is useful for precision procedures such as needle biopsy where the syringe needle tip may be moved in and out repeatedly to break up the target tissue with periodic small volume aspirations to acquire the tissue fragments.

An alternate method of use is shown in FIG. 3D in which a cradle grip (aka, hook or bucket grip) is utilized. In this method, the SSAA+syringe assembly is held in a cradle-grip with the SSAA mated in a superior position (over the top of the syringe). Typically, the SAA is attached to the syringe such that the volumetric gauge marks 119 are facing upward so as to facilitate viewing by the user. One or more fingers (two are shown) are placed on the syringe barrel and in contact with one or more of the syringe finger flanges. The thumb is placed on one or more of the thumb tabs located on the outside aspect of the linkage. The thumb can thereupon be moved proximally or distally to the barrel port to either eject or aspirate syringe contents (dashed double arrow). Such method of use may be preferred for low-angle procedures and/or situations in which desired ejection or aspiration volumes require greater precision than provided by the primary grips of FIGS. 3A and 3B, for example in dermatological procedures, shallow subdermal injections, and access to deeper structures inaccessible by high angle.

Other grip types are not shown but may also be useful. For example, a power grip (syringe in a I/O port down position, fingers curled around barrel, thumb on barrel flange) may be useful for subdermal procedures such as fine needle biopsy or when fingers-to-thumb spread is very large as may be the case when using large (60 cc and larger) syringes.

### Preferred Embodiments

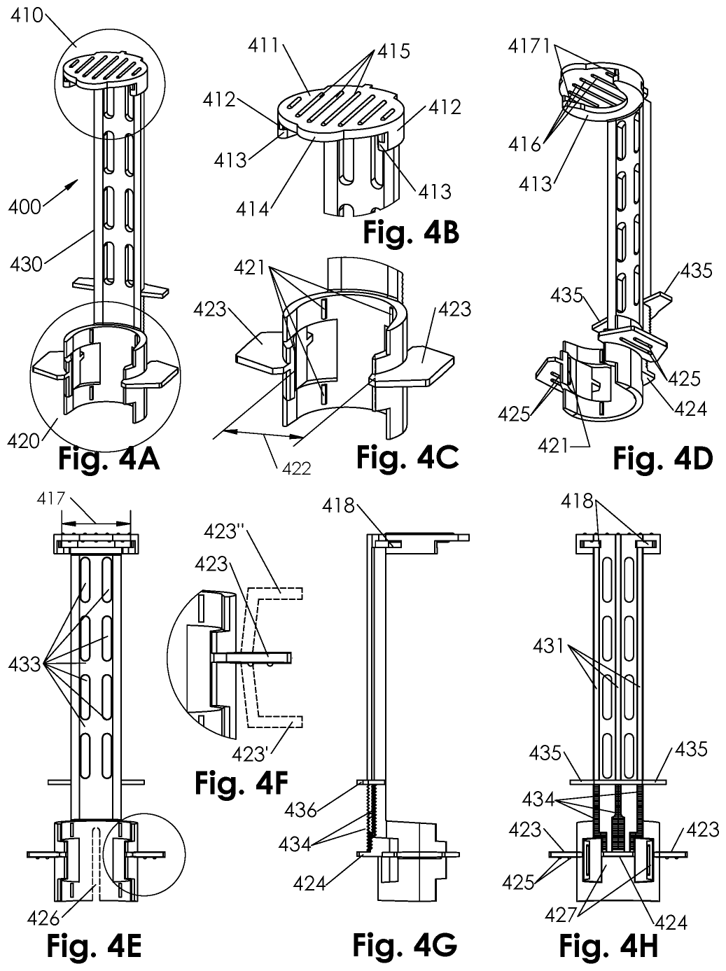

Various structures, features, and other modifications may be applied to the simplified embodiment 200 of FIG. 2A to achieve useful benefits and thus produce preferred embodiments. The benefits generally accrue to increased grip security, increased stiffness or structural integrity, ease of action during use, greater ease of attachment/detachment to the syringe, and improved moldability. The additional structures and features of a preferred syringe aspiration accessory (PSAA) 400 are described below and illustrated in FIGS. 4A-4G. As with the simple aspiration-assist accessory, the PSAA is comprised of three main sub-structures: A preferred plunger flange attachment 410, a preferred syringe barrel attachment 420, and a preferred linkage 430 unitary with and spanning the two attachment structures.

Preferred Plunger Flange Attachment (PPFA). The PPFA 410 of PSAA 400 is configured to allow simple yet secure fixation of the syringe aspiration device to the syringe plunger flange. It may be attachable and detachable, or only attachable (e.g., in cases where the device is to be disposed of after use along with the syringe). Typically, a clamp-style snap fitment is preferred as these are generally secure, do not require looping over the end of the syringe, and may be mated to the syringe at a range of locations along its length using simple linear motions. FIGS. 4A-B depict a preferred embodiment of a clamp-style annular snap-fit slot that is attached to the plunger flange using a linear lateral-to-axial motion. As with the SPFA, the PPFA comprises a top plate 411, side walls 412, and bottom plate 413. The top plate is generally planar and is typically formed in a disc-like shape, though other shapes (square, ovoid, rectangular, etc.) are possible. It may have a smooth or textured upper surface to improve grip. Conversely, it may be substantially peripheral, analogous to that depicted in the simple embodiment previously described, such being useful to avoid structures protruding from the top of the plunger flange, such as a thumb loop, should they be present. The top plate 4011 may also comprise other structures, including but not limited to a push tab 414, such being used to assist removal of the

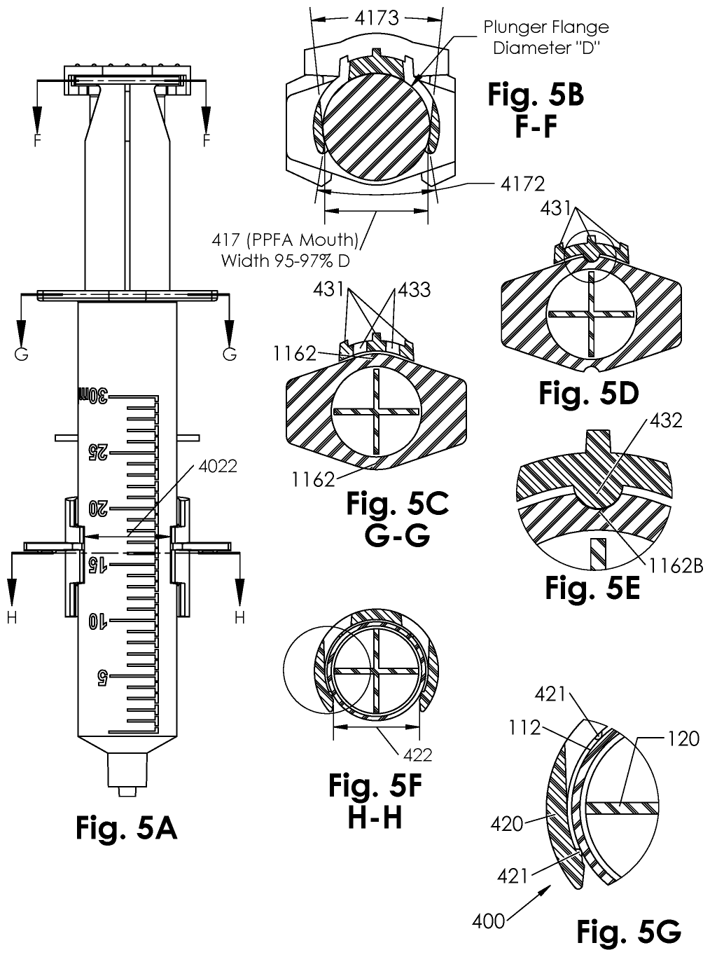

PPFA by providing the user with a contact-point to initiate disengagement of the attachment to the plunger flange 122. Analogous to the top plunger surface of many syringes, grip assist features 415 may be added to the PPFA top plate 4011, such features comprised of ridges, ribs, grooves, knurls, embossments, or other elements. Spacer ribs 416 may likewise be placed on the bottom surface of the plate to aid in accurately sandwiching the plunger top flange 122 between the top plate 411 and bottom plate 413 and thereby create a more secure fit. Together, the internal-facing features of the left and right side walls 412 define a PPFA mouth 417 (FIG. 4E) for introduction and retention of the plunger flange. The PPFA mouth typically comprises two vertical edges 4171 defining a minimum width, with walls tapering externally outward (to assist with lead-in of the plunger flange) thus forming a lead-in taper 4172, and walls tapering internally outward (to act as a retention undercut when the plunger flange is engaged) thus forming a retention taper 4173 (see FIG. 5B). To assist in molding, one or more upper molding windows 418 (FIG. 4H) may be placed in the PPFA. Such windows may allow use of telescoping shutoff elements to be positioned in the mold to assist in producing undercut part features without the use of side-acting elements (which must be moved prior to part ejection), specifically the inward-facing features of the PPFA mouth 417 (see FIG. 5B).

Preferred Syringe Barrel Attachment (PSBA). The PSBA 420 of preferred embodiment 400 is configured to allow easy yet secure engagement via a clamp-style snap fitment (aka, snap clamp) onto the syringe barrel. Once mated to the syringe barrel, the PSBA should slide smoothly up and down the barrel without binding, chattering, or unreasonable friction. As shown in the simple embodiment of FIG. 2A, the PSBA may be as simple as a cylindrical sleeve with an open side having a gap width slightly less than the OD of the barrel. To reduce friction as the PSBA slides up or down the barrel, barrel stand-offs 421 may be present at various locations on the inside surface of the PSBA sleeve such that the true contact area with the outside surface of the barrel 112 is minimal (as illustrated in FIG. 5F and 5G). Typically the inside diameter of the PSBA is slightly larger than the OD of the syringe barrel, while the inward extents of the barrel standoffs lie along an imaginary diameter which is slightly smaller than the OD of the barrel. In this fashion, the ID of the PSBA does not drag on the OD of the barrel but rather only the standoffs contact the OD of the barrel with minimal area, thereby allowing a smooth and low-friction action as the PSBA slides up or down the barrel. In addition to the semi-circular lands depicted in the preferred embodiment, the barrel stand-offs 421 may alternately be other low-profile geometries including but not limited to bumps, nubbins, thin flanges, ribs, or ridges, and may have simple or more complex cross-sectional shapes.

Nominally, the width of the PSBA mouth 422 (FIGS. 4C and 5F) is approximately 0.5 mm to 1.5 mm less than the OD of the barrel, or approximately a 2-6% diametral interference depending upon the stiffness of the material used to fabricate the SAA, its cross-sectional geometry, and the diameter of the plunger flange (the required interference to produce a secure fit will generally increase as the diameter of the plunger flange increases if the stiffness of the material used to fabricate the SAA is a constant). The PSBA mouth 422 is also useful for visualizing the piston 130 and its annular lips 132 within the syringe barrel and the volumetric gauge marks 119. Such direct visualization may be important to provide accurate volume control of the syringe contents both during aspiration and ejection. Force application features 423 (e.g., finger wings or flanges) are located on the outer surface of the PSBA and, similar to the analogous flange/wing structures 106 on the top collar of the syringe, project substantially laterally from the PSBA. The force application features may be configured as any of several geometries including but not limited to simple or tapered flanges, wings, or tabs; pins, dowels, or gussets; full or partial loops. Though the preferred embodiment is depicted as having two tapered flanges positioned on opposite sides of the syringe barrel, the force application feature may be substantially one sub-structure—e.g., a single semi-circular flange—or comprise three or more individual formations, depending upon the desired ergonomics and intended use. For example, FIGS. 4D and 4G-4H shows a preferred embodiment may have force application features comprising two opposing flanges 423 plus a single smaller thumb-assist flange 424 projecting from the “rear” aspect of the PSBA to provide a thumb force transmission feature for use when the syringe is cradled by the fingers in a low-angle “bucket handle” grip (i.e., low angle of entry to the skin). The preferred embodiment shows the force application features 424 positioned roughly at the horizontal midline of the PSBA—this positioning typically gives a good balance between ergonomics of use and force application to the PSBA body to achieve good sliding action. Other positions however may be useful, either toward the bottom end of the PSBA 423′ or upward toward the top 423″ (shown with dashed lines in FIG. 4F). The overall length of the PSBA will be defined by the diameter of the syringe barrel and should be the minimum length necessary to achieve a smooth up and down action (nonbinding) without off-axis tilting. For example, a 30 cc BD syringe with a barrel OD of approximately 24.2 mm uses a PSAA having a PSBA length of approximately 28 mm. It is generally preferred that the force application features 423 are positioned sufficiently above the bottom edge of the PSBA such that the user's fingers do not drag on the outer surface of the syringe barrel but rather rest upon the lower outside surface of the PSBA. Additional traction structures 425 including but not limited to small bumps, detents, grooves, and ridges may be added to the surfaces of the force application features to increase finger friction and grip security, as well as send tactile feedback to the user to indicate proper finger placement on the force application feature. It should therefore be noted that both the form and position of the force application features are variable depending upon the desired application, sizing, ergonomics, and manufacturing/molding considerations. One or more PSBA reliefs 426 (shown with dashed lines) may be placed through the PSBA wall to allow the snap-fit onto and off of the barrel to occur with lower force, as may be desired when using a stiff material to produce the SAA such as a metal or composite plastic. Relief structures include but are not limited to straight slots, t-slots, and key-hole slots. Furthermore, the relief may be of sufficient width to allow visualization of volumetric gauge marks 119 when the barrel is oriented in a horizontal position then using a cradle grip, such as depicted in FIG. 3C.

To assist in molding, one or more lower molding channels 427 (FIG. 4H), analogous to the upper molding windows 4018, may be embodied in the PSBA. Such windows would allow use of telescoping elements to be positioned in the mold to assist in producing undercut part features without the use of side-acting elements (which must be moved prior to part ejection), specifically the inward-facing features of the PSBA mouth 422, notably the front-side barrel stand-offs 421 (see FIG. 5G).

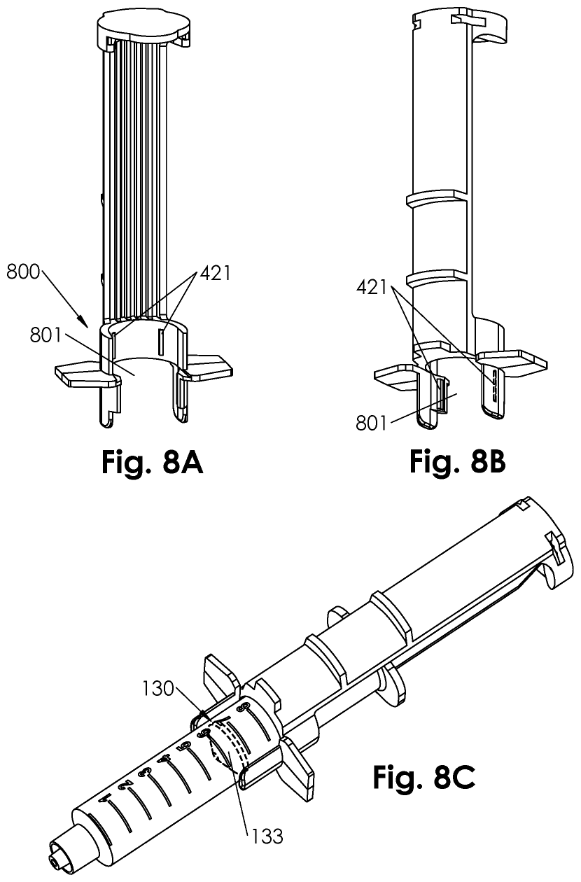

Preferred Linkage 430. The preferred linkage 430 of the PSAA is a substantially a single-body, linear compression element which ties/links the PPFA 410 to the PSBA 420. The linkage is positioned at or near the periphery of these elements such that it will reside upon or slightly lateral to the outer extent of the short annular flanges of the barrel top collar 1162, as shown in FIG. 5C. The linkage may assume a variety of cross-sectional (i.e., through a horizontal cross-sectional plane) forms including but not limited to round, ovoid, square, rectangular, arched, crescent, combinations thereof, or complex shapes. The linkage may be thick or thin; when thin, one or more linkage stiffening ribs 431 (FIGS. 4H, 5C, and 5D) may be employed to minimize bending during compression, such stiffening ribs typically running lengthwise along the outer surface of the linkage. Conversely, the outside surface may be substantially continuous with stiffening ribs projecting inward (as shown by reference in FIG. 8A). Other stiffening rib configurations such as cross-mesh, honey-comb, or other geometries may be used. A guide rib 432 may be positioned on the inside surface of the linkage to mate with a collar notch 1162B (FIGS. 1F and 5E) to provide an anti-rotation feature to the plunger (note: Collar notches 1162B may already be present on an as-molded OEM syringe or if desired may be included in the design of a custom syringe intended for channeling the guide rib 432); however, such a guide rib is not generally required if the inside surface of the linkage (or inside surfaces of the stiffening ribs in the case where stiffening ribs project inward) closely follows the annular flanges 1161 and 1162 of the top collar: A line-to-line or slightly offset sliding fit between the linkage and the top collar will commonly result in smooth action and minimal rotation of the plunger since the top collar finger flanges 1161 will constrain such rotation). Other features, such as linkage slots 433 (or depressions or thru-holes) may be placed in the linkage to reduce material usage and weight or assist moldability (FIG. 4E). The outside surface of the linkage 430, and/or stiffening flanges 4031 residing thereupon, may comprise low-profile traction grooves 434 (or ridges, bumps or knurls) to provide thumb purchase when using the syringe in a cradle grip (FIGS. 3C, 4G-4H)). In addition to the thumb-assist flange(s) 424 shown in FIGS. 4D, 4G, and 4H, traction grooves 434 allow proper application of thumb force without slippage to achieve precision aspiration and ejection, actions useful in dermatology and vascular access procedures, for example. One or more sets of starter wings 435 may be placed at any position along the linkage to provide users with smaller hands a reduced fingers-to-thumb span distance when the plunger is fully inserted. Once the plunger has been withdrawn to a comfortable degree, the user may move the fingers to the primary force application features 423 to continue aspiration. Starter wings 435 should be of a geometry to avoid interference as they slide past the barrel top collar during both aspiration and ejection. As with the force application features 423, the starter wings may take several forms, including flanges, pins, hooks, and other projection geometries. One or more secondary thumb-assist flanges 436 may positioned on the linkage to provide additional purchase when the syringe is used with a cradle grip or power grip. A set of starter wings may be contiguous with a secondary thumb-assist flange (as shown) or may be separate.

FIGS. 5A-5G show a front view and various sectional and detail views of the PSAA embodiment 400 mated to a typical syringe. FIG. 5B is a sectional view through line F-F, illustrating that the mouth of the PPFA receiver. The PPFA mouth 417 has a slightly smaller width than the OD of the plunger flange 122, typically 0.5 mm-1.5 mm smaller, or more generally about 3-5% smaller (Note: Larger syringes, such as 60 cc and above may require a greater difference between plunger flange OD and PPFA mouth width to achieve a secure fit for a given material stiffness and nominal wall thickness). The vertical height of the slot is typically 0-0.5 mm larger than the total height of the plunger flange (nominal thickness plus the height of any traction features 124 on top of the plunger top flange 122).

FIG. 5C is a sectional view along line G-G through the top collar 116 of the syringe barrel. It is seen that the inner surface of the linkage generally follows the lateral contour of the of the top collar to provide registration (resistance to turning of the linkage in relation to the central axis of the syringe). Alternately, as shown in FIGS. 5D-5E, a rib and groove tracking arrangement between one or more guide ridges 432 on the inner surface of the linkage and one or more notches 1162B in the short annular flanges of the top collar, thus providing additional resistance to turning (note: for clarity, linkage slots 433 are not shown).

FIGS. 5F-5G are a sectional view and associated detail along line H-H of FIG. 5A. These figures show the mating features between the PSBA and the outer surface of the syringe barrel. Specifically, barrel stand-offs 421 are seen to contact the outer surface of the syringe 112 with minimal contact area, thereby allowing a reduction of sliding friction during use.

### Alternate PPFA Embodiments

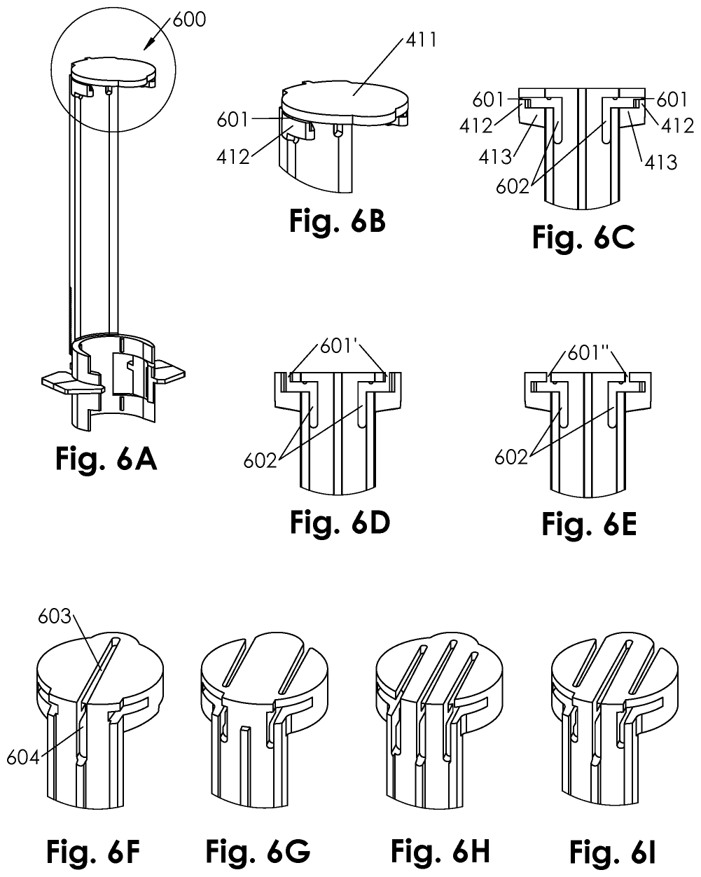

The preferred syringe aspiration accessory embodiments shown in FIGS. 4 and 5 depict a PPFA geometry that is optimized for a specific plunger flange diameter, or limited range of diameters. Alternate embodiments with the ability to attach to a wider range of plunger flange diameters may be desirable. FIGS. 6A-I depict various alternate-1 PPFA embodiments 600 capable of expanding to allow plunger flange diameters larger than the nominal diameter depicted in the prior figures. FIGS. 6A-6C show a PSAA with expansion reliefs 601 in the side walls 4012, the top plate 4011 remaining continuous. Laterally-transecting reliefs 601 allow the side walls 4012 and bottom plate 4013 to deflect outward to a greater degree than the prior PPFA geometry will allow (for a given material stiffness) and thus permit the introduction of and attachment to greater than nominal plunger flange diameters. Such laterally-transecting expansion reliefs may be positioned in various locations to permit adequate yet secure expansion, e.g. between the top plate and the side wall (as shown), or between the bottom plate and the side wall (not shown). To provide less resistance to deflection, linkage reliefs 602 may be provided to assist beam deflection and torsion. The width of the reliefs 601 and 602 will depend both upon the stiffness of the material used to fabricate the PSAA as well as the range of diameters the PPFA will be required to accommodate. Variations in the positioning of reliefs 601, 601′ and 601″, are seen in FIGS. 6D and 6E. The PPFA of FIG. 6D embodies reliefs 601′ in the top plate 4011 (rather than side walls 412) to allow full height of the side elements. The PPFA of FIG. 6E embodies reliefs 601″ in the top plate to allow sections of the top plate to be contiguous with the side walls and thereby define slots that engage the plunger flange from the top, bottom and sides.

FIGS. 6F-6I depict various PPFA embodiments 600 with partial top plate reliefs 603 that do not span the entirety of the top plate. Rear communicating partial top plate reliefs will typically have a contiguous linkage relief 604 to provide additional flexibility. As with the reliefs 601, 601′, and 601″ of FIGS. 6A-6E, such partial reliefs also provide for expansibility of the PPFA but may also provide a more secure fit since the top plate is spread in an accordion-like manner. The embodiments of FIG. 6A-6I depict a range of relief styles useful to provide expansibility of the PPFA but other relief features not shown may also provide benefits.

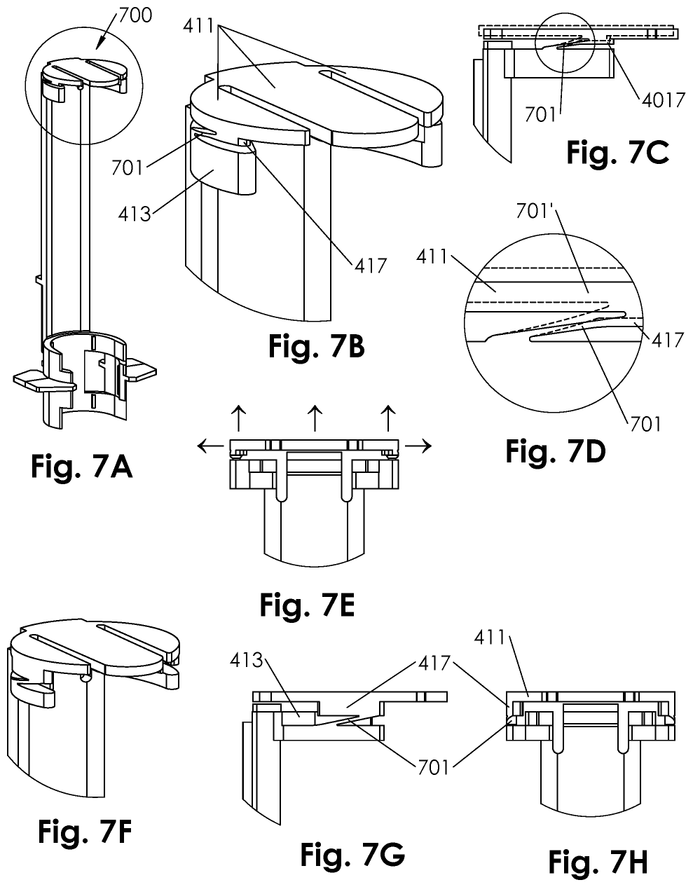

In addition to the alternate embodiments of FIGS. 6A-6I which allow use of a range of plunger flange diameters, FIGS. 7A-7E show alternate-2 PPFA embodiments 700 which allow use of a range of plunger flange thicknesses. FIG. 7A is a full image of such an embodiment with FIG. 7B a detail view of the PPFA. In this embodiment, the side walls (412 of FIG. 4B) have been relieved to produce leaf springs 701 which allow the top plate 411 to displace upward thereby accommodating a thicker plunger flange. FIG. 7C is a side view of the PPFA of this embodiment showing in dashed lines how the flexed leaf springs 701′ can bend to allow the top plate to move upward (and slightly backward). For this to work correctly, the top plate is relieved from the linkage, the top plate being connected to the bottom plate 413 only by the leaf springs 701 on either side. The PPFA mouth 417 on both sides is likewise relieved from the bottom plate to allow the top plate to shift upward as a thicker than nominal plunger flange is inserted. Because the PPFA for FIGS. 7A-7D utilize reliefs in the top plate and the linkage (analogous to FIG. 6G), it not only can accommodate a range of plunger flange thicknesses but also a range of plunger flange diameters, as shown by arrows in FIG. 7E. In this fashion the SAA can be attached to and function with several syringes within a volumetric class (as is available from different manufacturers) and thereby increase its applicability.

A modification to the alternate embodiment of FIGS. 7A-7E is shown in FIGS. 7F-7H. In the modified alternate embodiment, side walls 417 are relieved from bottom plate 413 and extended downward to a position level with or below the upper surface of the bottom plate. Leaf springs 701 again connect the side walls to the relieved bottom plate structure. The modified embodiment of FIGS. 7F-7H provide a slightly more constrained pocket for the OD of the plunger flange and therefore a more secure fit. As is the case with the embodiments depicted in FIGS. 6A-6I, the variants of FIGS. 7A-7E and 7F-7H can be molded with a straight-pull A-B mold (no side actions required), thus reducing mold cost and complexity.

### Alternate PSBA Embodiments

FIGS. 8A-C show an alternate-1 PSBA embodiment 800 which provides improved viewing of the syringe contents and plunger 130 when viewed from the rear aspect of the PSAA. In this alternate embodiment, the lower molding channels 427 of FIG. 4H have been widened and lengthened into a single expanded lower molding channel 801. FIGS. 8A and 8B show barrel standoffs 421 remain on the rear and front internal surfaces of the PSBA and that a substantially contiguous open space is formed which provides enhanced viewing from the rear aspect of this embodiment. FIG. 8C shows the alternate PSAA attached to a syringe (10 cc in this example).

The position of the syringe piston 130 and particularly the syringe bottom face 133 (dashed lines) is easily visualized through expanded lower molding channel 801, thus providing the user an unobstructed view of the syringe's contents and fill level. This embodiment is particularly useful for applications in which the PSAA and syringe are held in a cradle-grip, thereby presenting the rear aspect of the PSBA to the user's line of sight, as is common for use low angle dermatological procedures and fine-needle aspiration biopsy procedures. FIGS. 8A-8C depict a 10 cc syringe and appropriately sized PSAA, however this embodiment may be useful for smaller or larger syringe+PSAA combinations.

Methods of Use—PSAA

The methods of use of the PSAA and its variants are the same of those of the SSAA. The additional features of the PSAA provide performance, ergonomic, and fabrication advantages over the SSAA as well as additional or alternative usage modes. For example, starter wings 435 shown on the PSAA but not the SSAA allow users with smaller hands to comfortably initiate aspiration without fully extending the fingers and thumb; nevertheless such features as seen on the PSAA may generally be applied to the SSAA as well.

Materials and Manufacturing Processes.

The specific dimensions of the SAA are defined primarily by the size of the syringe to which it is applied and the stiffness of the material used to produce the SAA. For example, the Linkage 430 may be made narrower and thinner and may obviate the need for stiffening ribs 431 if the material used to fabricate the SAA is relatively stiff, such as glass-reinforced nylon or a metal such as aluminum. Conversely if the the material is soft or semi-soft, such as clarified polypropylene (the material most commonly used in the production of disposable syringes), the Linkage will need to be wider/thicker and stiffening ribs may be required to minimize bending. Lubricity is another consideration: The material selected to produce the SAA should have good lubricity against the material used to mold the syringe barrel and plunger. If sliding friction is too high, one or both of the PPFA and the PSBA may bind upon attachment or detachment; likewise, the PSBA may bind on the syringe barrel when sliding during use. Disposability versus durable-use should be considered: For example, if the syringe used is disposable and produced from polypropylene, it may be useful to produce the SAA from a similar material to facilitate recycling or disposability. Durable-use SAAs may utilize more expensive, durable materials such as engineering plastics (e.g. Nylon, polycarbonate), composites (e.g. glass-fiber reinforced ABS), and metals. Whether the SAA will be sterilized and the the method of sterilization must also be considered—certain materials though mechanically suitable may not permit necessary modes of sterilization, such as wipe-down with ethanol, UV light, autoclave, gamma, or e-beam irradiation due to material degradation. The method of manufacture will likewise be contingent upon multiple factors, both performance and economic. Most commonly, the SAA will be manufactured via injection molding of a thermoplastic material. These materials may be filled (glass, mineral, or carbon fiber or particle) or unfilled and include put are not limited to polypropylene, polyethylene, nylon (polyamide), polycarbonate, ABS, or polystyrene. The SAA may also be produced via additive methods such as 3D printing or subtractive methods such as machining. Thermosetting plastics may also be used to mold the SAA and may be produced via injection molding, compression molding, or casting. Metals such as aluminum, steel, or titanium may be used and finished parts can be produced via machining, casting, metal injection molding, or 3D printing. It may be desirable to apply a secondary step during production, such as anodization of aluminum, to increase surface durability, improve aesthetics, or reduce friction when engaging and sliding upon the syringe.

As stated previously, features may be added to the various sub-structures of the SAA to assist production. Of primary example, upper windows 418 and lower molding channels 427 may be disposed through the PPFA and PSBA surfaces to allow for telescoping mold elements to extend through the part. Telescoping mold elements may allow the use of simple two-sided “straight-pull” molds for molding the SAA, thus eliminating the need for expensive and complicated side-acting components to mold the undercut snap-fit features. Straight-pull molds are generally more simple, lower in cost, more durable, and more reliable than molds with side-action components. Other features, notably drafted surfaces, may be provided within the SAA geometry to ease part ejection and attain high quality surface finishes.

The additional features and structures of the various preferred embodiments may be combined in ways to achieved desired outcomes; not all features and structures described must necessarily be applied but rather those useful for a particular outcome or functional capability. For example, it may be useful to use a non-expandable PPFA (as shown in FIGS. 4A-H and 5A-G) when the SAA is intended for use with a specific syringe. Conversely, if the SAA is intended to be used with a range of different syringes within a size class (e.g., 30 cc or 60 cc) from various manufacturers, it will be useful to use an expandable PPFA structure (as shown in FIGS. 6A-I) to allow attachment to a larger range plunger top flange diameters.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

I claim:

1. A unitary syringe accessory device configured to attach to a typical syringe, comprising:
A syringe plunger flange attachment means, and
a syringe barrel attachment means, and
at least one force application structure disposed upon and integral to said syringe barrel attachment means, and
a linkage connecting said barrel attachment means and said plunger flange attachment means,
wherein the syringe accessory provides a mechanical advantage when aspirating contents into the syringe during single-handed operation.

2. The plunger flange attachment means of claim 1, comprising:
a top plate, and
a discontinuous bottom plate, and
at least one discontinuous side wall,
wherein said top plate, bottom plate, and side wall are integral and substantially contiguous,
such that said plunger flange attachment comprises a pocketed receiver for fitment to a syringe plunger flange.

3. The top plate of claim 2 comprising a substantially discontinuous ring-like element, wherein the top plate is at least partially open such that thumb or finger force may be applied to the top surface of the plunger flange to induce motion of the plunger into the syringe barrel and thereby eject contents out of the syringe.

4. The top plate of claim 2, comprising a substantially continuous planar element, whereupon thumb or finger force may be applied to the top surface of the top plate to induce motion of the plunger into the syringe barrel and thereby eject contents out of the syringe.

5. The substantially continuous planar element of claim 4, comprising a radially protruding element positioned substantially on the front periphery of the substantially continuous planar element, whereupon force may be applied to assist removal of the plunger flange attachment means from the syringe plunger.

6. The substantially continuous planar element of claim 4, comprising reliefs to allow lateral expansion, whereby plunger flanges in a range of diameters may be inserted into and secured by the plunger flange attachment means.

7. (canceled)

8. (canceled)

9. The at least one discontinuous side wall of claim 2, comprising reliefs through said at least one side wall, whereby syringe plunger flanges in a range of thicknesses may be inserted into and secured by the plunger flange attachment means.

10. The plunger flange attachment means of claim 1, comprising a slotted receiver of an open-sided substantially c-form cross-sectional shape, wherein the opening of said slotted receiver has a maximum open-side qap width less than or equal to the outside diameter of the syringe plunger flange, and whereby said plunger flange attachment means may be fitted to the flange element of a syringe plunger, and whereby force may be transmitted to said syringe plunger flange in both axial directions.

11. (canceled)

12. The syringe barrel attachment means of claim 1, comprising an open-sided sleeve structure, whereby said sleeve structure can be snap-fitted to the cylindrical outside surface of a syringe barrel using a substantially lateral to medial motion.

13. The at least one force application structure of claim 1 comprising but not limited to the class of flanges, wings, tabs, ribs, gussets, bars, pins, posts, or other protrusions, whereupon finger or thumb force may be applied in a substantially normal direction to a surface thereof, whereby movement of the syringe plunger outward from or inward into the syringe barrel may be actuated, and thereby induce the aspiration or ejection of contents into or out of the syringe, respectively.

14. (canceled)

15. The open-sided sleeve structure of claim 12, comprising stand-off structures on the inside surface of said open-sided sleeve structure, wherein contact area and friction between the sleeve structure and the syringe barrel are reduced.

16. The open-sided sleeve structure of claim 12, comprising at least one relief extending upward from the bottom edge of said open-sided sleeve structure and through the thickness of the sleeve, whereby said open side of the sleeve may be more easily expanded during engagement to and removal from a syringe barrel.

17. (canceled)

18. The at least one channel of claim 17, comprising at least one opening through the sleeve structure, wherein said opening is substantially contiguous and in communication with the bottom edge of the sleeve structure, and whereby visibility of the syringe piston is substantially unobstructed by the sleeve structure.

19. (canceled)

20. The linkage of claim 1, comprising a substantially continuous linear structure.

21. The linear structure of claim 20, comprising two ends, wherein a first end is located on or proximal to the periphery of the barrel attachment means and a second end is located on or proximal to the periphery of the plunger flange attachment means.

22. The linear structure of claim 20, substantially comprising any of the cross-sectional shapes including but not limited to squares, rectangles, circles, ellipses, I-forms, T-forms, C-forms, crescents, and other complex forms.

23. The linear structure of claim 20, comprising a cross-sectional shape, wherein the inside profile of the linkage is substantially similar to the outside profile of the top collar of the syringe barrel, and whereby said linkage may track closely to and without interference with said top collar.

24. The linear structure of claim 20, comprising at least one stiffening rib oriented length-wise on the outer surface of the linkage, wherein resistance to bending is increased.

25. The linear structure of claim 20, comprising at least one stiffening rib oriented length-wise on the inner surface of the linkage, wherein resistance to bending is increased.

26. (canceled)

27. The linear structure of claim 20, comprising at least one through-hole positioned between the outer and inner surfaces of said linkage, wherein material usage and part weight are reduced.

28. (canceled)

29. The linear structure of claim 20 comprising at least one thumb engagement feature on the outer surface proximal to said first end of said linear structure, and whereupon thumb force may be applied in either axial direction when the syringe is grasped in a cradle-grip orientation to facilitate aspiration or ejection of syringe contents, said at least one thumb engagement feature including but not limited to the class of flanges, wings, tabs, ribs, gussets, gnurling, nubbins, posts, embossments, or other protrusions.

30. (canceled)

31. The linear structure of claim 20, comprising at least one tracking rib oriented length-wise on the inner surface of said linkage, wherein tracking on at least one associated detent in the syringe barrel collar is facilitated, and whereby rotational orientation between the syringe and the syringe accessory device is maintained.

32. (canceled)

33. A method for utilizing a unitary syringe accessory device comprising a syringe plunger flange attachment means, a syringe barrel attachment means, at least one force application means disposed upon and integral to said syringe barrel attachment means, and a linkage connecting said barrel attachment means and said plunger flange attachment means, consisting of:
Laterally aligning the central axis of the accessory device to the central axis of the syringe, and
approximating the accessory device to the syringe using a substantially lateral-to-medial motion, and
snap-fitting said syringe barrel attachment means of the accessory device to the syringe barrel, and
snap-fitting said plunger flange attachment means of the accessory device to the syringe plunger flange,
wherein the syringe barrel comprises at least one flange for application of manual force.

34. The method of claim 33, further consisting of:
Placing the two of the user'fingers individually on the inferior surfaces of at least one force application structure of said syringe barrel attachment means, and
placing the user's thumb on a superior surface of the at least one barrel flange of the syringe, and
approximating the user's index and middle fingers toward the user's thumb,
whereby the syringe plunger is progressively retracted from the syringe barrel, and
whereby negative pressure within the syringe barrel is produced and contents may be aspirated into the syringe.

35. The method of claim 33, consisting of:
Orienting the syringe and attached accessory device in a substantially horizontal position with the accessory device superiorly positioned to the syringe, and
grasping the syringe and attached accessory device in the user's hand using a cradle-grip, and
placing the user's thumb on at least one force application structure disposed upon and integral to the outer surface of said accessory device proximal to the junction of said barrel attachment means and said linkage,
whereby contents may be aspirated into the syringe by approximating the tip of the thumb toward the base of the thumb, and
whereby contents may be ejected from the syringe by extending the tip of the thumb away from the base of the thumb.

36. (canceled)

37. (canceled)

38. (canceled)

39. (canceled)
